(19) United States
(12) Patent Application Publication
(10) Pub. No.: US 2014/0163365 A1
(43) Pub. Date: Jun. 12, 2014

(54) Hand Controller Support Assembly for a Fluid Delivery System

(71) Applicants: Michael T. Shaffer, Georgetown, PA (US); Patrick B. Campbell, Pittsburgh, PA (US)

(72) Inventors: Michael T. Shaffer, Georgetown, PA (US); Patrick B. Campbell, Pittsburgh, PA (US)

(73) Assignee: Medrad, Inc., Indianola, PA (US)

(21) Appl. No.: 13/761,470

(22) Filed: Feb. 7, 2013

Related U.S. Application Data
(60) Provisional application No. 61/734,707, filed on Dec. 7, 2012.

(51) Int. Cl. A61M 5/00

(52) U.S. Cl. CPC A61M 5/007; A61M 5/008
USPC 600/432; 248/205.1

(57) ABSTRACT

A fluid delivery system is provided, including a fluid injector head, a hand controller electronically interfaced with the fluid injector head, and a hand controller support assembly for supporting the hand controller. The hand controller support assembly includes a first component having a base portion and two outward extending support arms to support the hand controller and a second component adapted for fixation to a surface. The first component may be rotationally connected to the second component such that the first component may automatically reposition relative to the second component under the force of gravity to maintain the hand controller in an upright position and supported by the support assembly.

### CROSS REFERENCE TO RELATED APPLICATIONS

This application claims the benefit of U.S. Provisional Application No. 61/734,707, filed Dec. 7, 2012. This application incorporates by reference the subject matter of: U.S. Pat. No. 7,879,008 (Haury et al.); application Ser. No. 12/265,060 (Schriver et al.) published as US 2010/0114040; and International Application No. PCT/US12/37491 (WO 2012/155035).

### BACKGROUND OF THE INVENTION

1. Field of the Invention

This disclosure relates generally to control devices for controlling operation of fluid-supplying machines or apparatus used in medical procedures such as angiography and, further, to hand-held control devices for controlling the flow rate of fluids, such as contrast media and/or common flushing agents, injected into a patient during medical procedures, such as angiography.

2. Description of Related Art

As an example, angiography is a procedure used in the detection and treatment of abnormalities or restrictions in blood vessels. During angiography, a radiographic image of a vascular structure (i.e., blood vessel) is obtained by injecting radiographic contrast material, also referred to as contrast media, through a catheter into a vein or artery. X-rays are passed through the region of the body in which the contrast media is concentrated. The X-rays are absorbed by the contrast material, causing a radiographic outline or image of the blood vessel containing the contrast media. The X-ray's images of the blood vessel filled with the contrast media are usually recorded onto film or videotape and are displayed on a fluoroscope monitor.

Many angiographic procedures, in particular coronary angiography and especially coronary vascular interventional procedures such as angioplasty, require frequent intermittent injections of contrast media. The contrast media is administered in varying volumes as well as modulated strengths and time durations. The intermittent contrast media injections are critical for optimal positioning of guiding catheters at the targeted blood vessels, positioning of guide wires to and through the targeted areas during catheter interventions (i.e., percutaneous transluminal coronary angioplasty), and for assessment of the results of such interventional procedures.

During angiography, after a physician places the angiographic catheter into a vein or artery, the angiographic catheter is connected to either a manual or an automatic contrast media injection mechanism. A typical manual contrast media injection mechanism includes a syringe and a catheter connection. The user of the manual contrast media injection mechanism adjusts the rate and volume of injection by altering the manual actuation force applied to the plunger of the syringe.

Automatic contrast media injection mechanisms typically involve a syringe connected to a linear actuator. The linear actuator is connected to a motor which is controlled electronically. The operator enters into the electronic control a fixed volume of contrast media and a fixed rate of injection. There is typically no interactive control between the operator and the mechanism, except to start or stop the injection. A change in flow rate occurs by stopping the mechanism and resetting the parameters.

Recent improvements in the radiographic imaging field have attempted to apply software and hardware interfaces to automatic contrast media injection mechanisms to provide variable flow rate and fixed flow rate modes to the operator. Additionally, the delivery of common flushing agents, such as saline, may also be controlled using the software/hardware interfaces. One such angiogaphic control device is disclosed in U.S. Pat. No. 5,515,851 to Goldstein. The Goldstein patent discloses the use of a microchip control device in the form of an angiographic control pad device designed to facilitate finger touch modulation of flow rate, volume, and duration of contrast media injection into a patient during an angiographic procedure. The control finger pad device allows the operator to control the aforementioned parameters during an injection procedure by altering the duration and extent of fingertip depression on the finger pads.

Another control device used to provide variable flow rate control to an operator of an automatic contrast media injection mechanism is disclosed in U.S. Pat. No. 5,916,165 to Duchon et al. This reference discloses a hand-held pneumatic control device that interfaces with and controls a fluid supply or injection mechanism. The hand-held control device is further adapted to control dispensement of saline injected into the patient during the angiographic procedure. The hand-held control device is generally adapted to be responsive to fluid pressure within the device. The control device includes a pressure control member adapted to selectively change fluid pressure within the pressure control member based on inputs from the operator. In one embodiment, the control device is provided with one or more internal air bladders having a volume that selectively adjusts to change the fluid pressure within the air bladders based on operator inputs. Internal sensors are provided to monitor the volume changes of the air bladders, and generate control signals based on the volume changes.

U.S. Pat. No. 5,988,587, also to Duchon et al., discloses another version of a hand-held control device for an automatic contrast media injection mechanism. This reference discloses a hand-held control device that includes two opposing and spaced-apart handles. A resilient attachment member connects the two handles. The resilient attachment member is configured to allow the first handle to move with respect to the second handle in response to operator inputs. The control device includes a sensor attached to the first handle for producing a variable control signal indicative of the distance between the first handle and the second handle.

Yet another hand-held control device is disclosed in U.S. Pat. No. 6,221,045 to Duchon et al. This reference discloses a hand-held control device that generates a control signal that is continuously variable according to continuously varying movement of a user's hand on the hand-held control. The control signal is continuously variable and sustainable at any value between preset maximum and minimum values corresponding to maximum and minimum contrast media discharge flow rates.

### SUMMARY OF THE INVENTION

In view of the foregoing, it is desirable to provide a hand controller support for association with a power fluid injector head that can assist holding the hand controller in between fluid injection procedures and help retain the sterility of the hand controller and/or a disposable sheath associated with the hand controller, which covers the hand controller during a fluid injection procedure. In one embodiment, a support assembly is provided as a multi-part component, such as a two-part component, which may be “snap-fitted” together and may be adapted so as to rotate or swivel in conjunction with conventional rotation of the fluid injector head. A desirable result of this rotational or swivel feature of the support assembly is that the support assembly can rotate with the fluid injector head as it rotates and the rotational or swivel feature maintains the hand controller in the support assembly so that the hand controller does not fall out of the support assembly. The support assembly may be installed and attached to the fluid injector head by simple mechanical fasteners that attach the support assembly to the back of the fluid injector head. The portion of the support assembly that holds the hand controller may be designed in such a way that the hand controller is easily snapped in and out of the support assembly.

In one exemplary embodiment, a hand controller support assembly is provided, comprising a first component comprising a base portion and two outward extending support arms to support the hand controller and a second component adapted for fixation to a surface. The first component may be rotationally connected to the second component such that the first component may automatically reposition relative to the second component under the force of gravity to maintain the hand controller in an upright position and supported by the support assembly.

The second component may comprise an L-shaped body comprising a first leg and a second leg defining approximately a 90° angle between one another. The first leg may be longer than the second leg. The first leg may define a longitudinal slot between two arm portions, with the longitudinal slot formed with a circular recess to receive a support post extending from the base portion of the first component to enable rotational movement of the first component relative to the second component. The circular recess may be accessed via a V-shaped notch defined by opposed tapered ends of the arm portions.

The second component may comprise an L-shaped body comprising a first leg and a second leg, and wherein the second leg defines a slot for a fastener to attach the second component to the fixation surface. The second leg may further comprise stabilizing legs adapted to contact the fixation surface.

A support post may extend from the base portion of the first component. The second component may comprise an L-shaped body comprising a first leg and a second leg, and the first leg may define a longitudinal slot between two arm portions. The longitudinal slot may be formed with a circular recess to receive the support post to enable rotational movement of the first component relative to the second component. The support post may comprise a button element to limit axial movement of the support post in the circular recess.

In another embodiment, a fluid delivery system is provided, comprising a fluid injector head, a hand controller electronically interfaced with the fluid injector head and/or a graphical user interface associated with the injector head, and a hand controller support assembly for supporting the hand controller. The hand controller support assembly comprises a first component comprising a base portion and two outward extending support arms to support the hand controller and a second component adapted for fixation to a surface. The first component may be rotationally connected to the second component such that the first component may automatically reposition relative to the second component under the force of gravity to maintain the hand controller in an upright position and supported by the support assembly.

The second component may comprise an L-shaped body comprising a first leg and a second leg defining approximately a 90° angle between one another. The first leg may be longer than the second leg. The first leg may define a longitudinal slot between two arm portions, with the longitudinal slot formed with a circular recess to receive a support post extending from the base portion of the first component to enable rotational movement of the first component relative to the second component. The circular recess may be accessed via a V-shaped notch defined by opposed tapered ends of the arm portions.

The second component may comprise an L-shaped body comprising a first leg and a second leg, and wherein the second leg defines a slot for a fastener to attach the second component to the fixation surface. The second leg may further comprise stabilizing legs adapted to contact the fixation surface.

A support post may extend from the base portion of the first component. The second component may comprise an L-shaped body comprising a first leg and a second leg, and the first leg may define a longitudinal slot between two arm portions. The longitudinal slot may be formed with a circular recess to receive the support post to enable rotational movement of the first component relative to the second component. The support post may comprise a button element to limit axial movement of the support post in the circular recess.

Further details and advantages will be understood from the following detailed description read in conjunction with the accompanying drawings.

### BRIEF DESCRIPTION OF THE DRAWINGS

FIG. 1 is a perspective view of a mobile fluid delivery system comprising a power fluid injector head according to one embodiment.

FIG. 2 is a perspective view of the fluid delivery system mounted to the rail of an examination table and comprising a power fluid injector head.

FIG. 3 is perspective view of a hand held control device in accordance with one embodiment and adapted to electronically interface with the fluid delivery system of FIGS. 1-2.

FIG. 4 is a perspective view of a support assembly used to support the hand held control device of FIG. 3 in the fluid delivery system of FIGS. 1-2.

FIG. 5 is a perspective and exploded view of the support assembly shown in FIG. 4.

FIG. 6 is a perspective view of a first component of the support assembly shown in FIG. 4.

FIG. 7 is a perspective view of a second component of the support assembly shown in FIG. 4.

FIG. 8 is a plan view of the second component shown in FIG. 7.

FIG. 9 is a plan view of the second component shown in FIG. 7.

FIG. 10 is a perspective view showing the support assembly connected to a fluid injector head of the fluid delivery system of FIGS. 1-2, and the support assembly supporting the hand controller of FIG. 3.

### DESCRIPTION OF THE PREFERRED EMBODIMENTS

For purposes of the description hereinafter, spatial orientation terms, as used, shall relate to the referenced embodiment as it is oriented in the accompanying drawing figures or otherwise described in the following detailed description. However, it is to be understood that the embodiments described hereinafter may assume many alternative variations and configurations. It is also to be understood that the specific components, devices, features, and operational sequences illustrated in the accompanying drawing figures and described herein are simply exemplary and should not be considered as limiting.

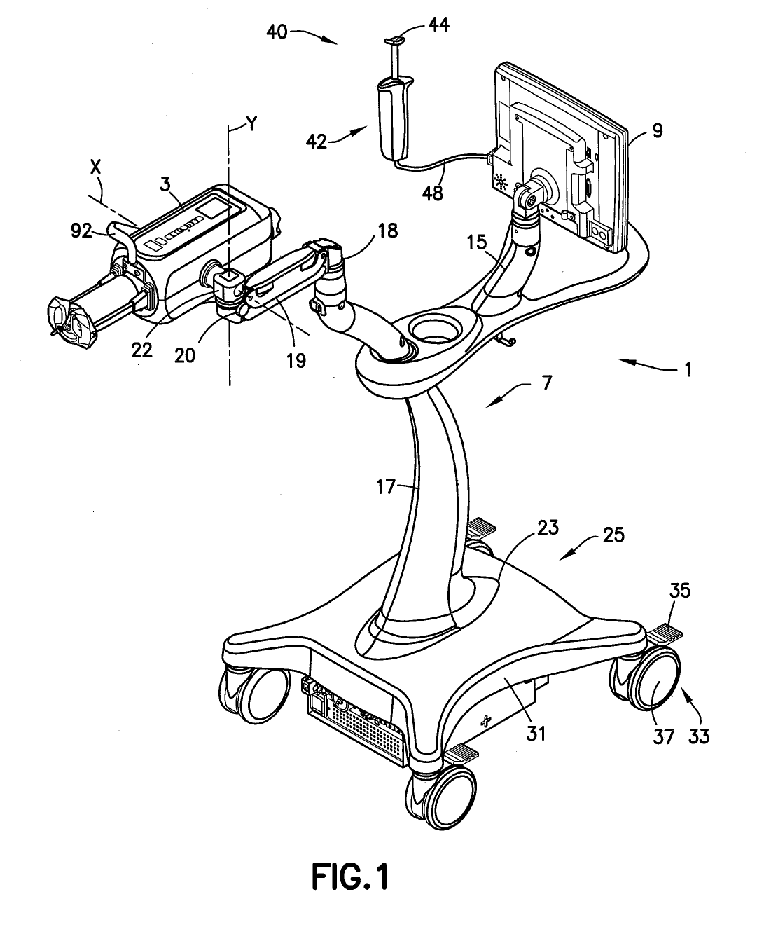

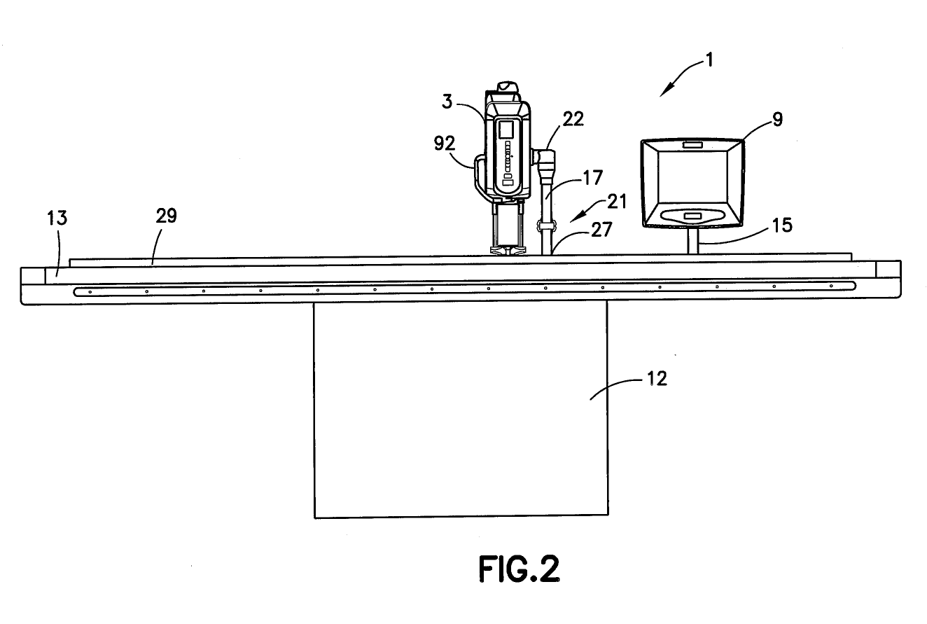

With reference to FIGS. 1-2, a fluid delivery system, generally denoted as reference numeral 1, includes a power fluid injector head 3 for delivering a fluid to a patient 5; a mounting structure 7 pivotally connected to the fluid injector head 3 and configured to support the fluid injector head 3; and a control system, including a display control unit (DCU) 9, operationally coupled to the fluid injector head 3 for controlling an injection procedure. Further details of the fluid injector head 3 are disclosed in U.S. Pat. Nos. 7,326,186; 7,549,977; 7,556,619; 7,563,249; and 7,611,503 and United States Patent Application Publication No. 2008/0086087, which are hereby incorporated by reference in their entirety. The foregoing references provide further and/or alternative embodiments for the fluid injector head 3. However, a desired embodiment of the fluid injector head 3 for the purposes of this disclosure are presented in international Application No. PCT/US12/37491 (WO 2012/155035), previously incorporated by reference.

The DCU 9 includes a color liquid crystal display (LCD) screen with a touch screen overlay that is interfaced to an internal computer board. The DCU 9 is responsible for providing a graphical user interface (GUI) to the user and allows information to be input via the touch screen to the control system. In addition, the fluid delivery system 1 can support multiple display DCUs, such as auxiliary DCUs. When multiple DCUs are incorporated into the fluid delivery system 1, the multiple DCUs are not operated in a master/slave configuration. Instead, a user may interface with any of multiple input devices such as the display controls (discussed hereinafter) of the fluid injector head 3 or any of the multiple DCUs 9. While the user is interacting with one of these input devices, control inputs from the others are ignored (i.e., locked). However, their display outputs can be updated by the active input device. When the user has returned to the Home screen of a DCU, the lock on all input devices is removed. Another way in which control may be given up and the lock removed, is through the use of timeouts. If the interface device is not interacted with by the user for a period of time (5 seconds for fluid injector head inputs, 30 seconds for DCU inputs), an audible or visual indicator is asserted and control is given up by the active input device.

FIG. 1 illustrates the fluid delivery system 1 provided as a mobile unit and FIG. 2 illustrates the fluid delivery system 1 as being mounted to an examination table 13. In each configuration, the mounting structure 7 of the fluid delivery system 1 includes a first support arm 15 extending from a support column 17 for supporting the DCU 9. A second support arm 19 extends from the support column 17 and generally supports the fluid injector head 3. The second support arm 19 has a first end 18 pivotally coupled to the support column 17 and a second end 20 pivotally coupled to a knuckle 22 of the fluid injector head 3.

In the configuration shown in FIG. 2, the support column 17 is associated with a rail interface 21 which is generally adapted to attach the fluid delivery system 1 to a hospital bed or an examination table 13 supported by a stand 12. Alternatively, and as shown in FIG. 1, the support column 17 may include a pedestal interface 23 for attaching the fluid delivery system 1 to a movable pedestal 25. The fluid delivery system 1 may be configured to be attached to the examination table 13 or the movable pedestal 25 to provide the maximum amount of flexibility and ease in utilizing the fluid delivery system 1. Thus, when the fluid delivery system 1 is mounted to the examination table 13, a rail mount 27 is attached to a rail 29 of the examination table 13. This allows the rail interface 21 to be removably attached to the rail mount 27. Thus, the rail mount 27 indirectly supports the DCU 9 and the fluid injector head 3. In an alternative embodiment, only the fluid injector head 3 is indirectly supported by the rail mount 27, and an additional rail mount (not shown) is utilized to independently support the DCU 9 at a different location on the rail 29 of the examination table 13.

As shown in FIG. 1, the movable pedestal 25 provides mobility to the fluid delivery system 1 and height adjustability features. The movable pedestal 25 includes a base 31 for holding loose components related to the fluid delivery system 1 and the power cables associated therewith. The base 31 may also include a power socket (not shown) that interfaces with the power cables (not shown) within the base 31. Thus, a single external power cable (not shown) may be plugged directly into the power socket (not shown) to provide sufficient power for operation of the entire fluid delivery system 1. The movable pedestal 25 may also include a plurality of casters 33 having lockable brakes 35 and wheels 37. It is to be understood that the aforementioned configurations are for exemplary purposes only and are not to be considered as limiting the placement and positioning of the fluid delivery system 1. A handle 92 is also positioned on the fluid injector head 3 for repositioning or transporting the fluid delivery system 1.

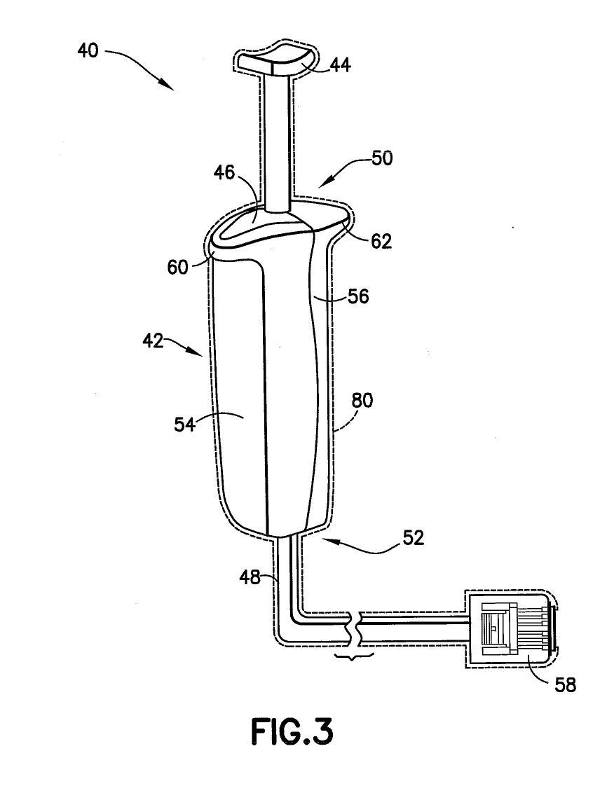

With further reference to FIG. 3, a hand held control device 40 according to one embodiment is shown. The control device 40 (hereinafter “hand controller 40”) is desirably configured to be hand-held. However, this form of the hand controller 40 is merely exemplary, and the hand controller 40 may be provided as a foot-controller or a robotic actuated device, as examples, or simply as an electronic console with one or more actuating devices, such as buttons, joysticks, and like elements. With general reference to FIG. 3, the externally visible components of the hand controller 40 generally include a housing 42, a first actuator 44 associated with the housing 42, a secondary actuator 46 also associated with the housing 42, and a cable 48 extending from the housing 42. The placement of these and any other components of the hand controller 40 are with reference to the presently illustrated embodiment and should not be construed as limiting.

Generally, the first actuator 44 and the secondary actuator 46 are disposed at a top end 50 of the housing 42, and the cable 48 extends from a bottom end 52 of the housing 42. The housing 42 may have an ergonomic shape, so that the hand controller 40 may be comfortably held in either the left or right hand by a user, and to allow for single-handed operation thereof, as generally disclosed in application Ser. No. 10/237,139, filed on Sep. 6, 2002, assigned to the same assignee as the present application, the disclosure of which is incorporated herein in its entirety. The housing 42 includes at least a first portion and a second portion, such as a left side or portion 54 and a right side or portion 56, respectively. However, the housing 42 may include any number of pieces or components and is generally intended to be a multi-piece structure. The housing 42 is desirably formed of plastic material, such as a suitable medical-grade plastic material. Inexpensive materials may be used for the housing 42 and the other components of the hand controller 40 so that the hand controller 40 may be a disposable item, disposed of, for example, after a preset number of procedures are conducted using the fluid delivery system 1. The housing 42 is formed to enclose and support the internal components of the hand controller 40 to be discussed herein. The hand controller 40 weighs in the range of about 0.25 to 1 pound, so that the hand controller 40 may be comfortably manipulated by an operator for extended periods of time without fatigue. The two side portions 54, 46 may be formed with opposing arms 60, 62 which provide supporting locations for the hand controller 40 to rest against the user's hand as the user grasps the hand controller 40 during operational use.

The cable 48 is generally adapted to transmit input commands in the form of digital values from the hand controller 40 to the fluid injection system 1. The cable 48 may be any suitable type of cable adapted to digitally transfer the digital values to the fluid delivery system 1. For example, the cable 48 may be any suitable multiple-strand wiring cable, such as 6-pin phone cable. The cable 48 terminates in a connector 58 which is adapted to operatively and removably associate the hand controller 40 with the fluid injector head 3 of the fluid delivery system 1, and electronically interface the hand controller 40 with the fluid injector head 3 and/or the DCU 9 as shown in FIG. 1, whereby manual inputs to the hand controller 40 are electronically transmitted via the DCU 9 to control operation of the fluid injector 3. The connector 58 may alternatively be plugged into a suitable electronic port (not shown) on the fluid injector head 3 to establish electronic communication with the fluid injector head 3 and DCU 9, as will be appreciated by those skilled in the computer arts. The connector 58 may be, for example, an RJ11 connector with six contacts which allows the end of the cable 48 distal or remote from the hand controller 40 to have a positive locking electrical connection with any desired electronic port of the fluid delivery system 1. Further details of the hand controller 40 may be found in U.S. Pat. No. 7,879,008 (Haury et al.) and application Ser. No. 12/265,060 (Schriver et al.) published as US 2010/0114040, which were previously incorporated by reference.

In order to maintain sterility and prevent contamination, the hand controller 40 may utilize a sterile sheath 80, which is configured as a generally form-fitting envelope enclosing at least the housing 42 of the hand controller 40. The sterile sheath 80 may enclose the first actuator 44 and cable 48 as shown in dotted lines in FIG. 1. The sterile sheath 80 may be transparent and is not intended to impair any operator functions of the hand controller 40. This optional sterile sheath 80 may be made of inexpensive material, desirably plastic, and disposed after each use of the hand controller 40, extending the usable disposable life of the hand controller 40.

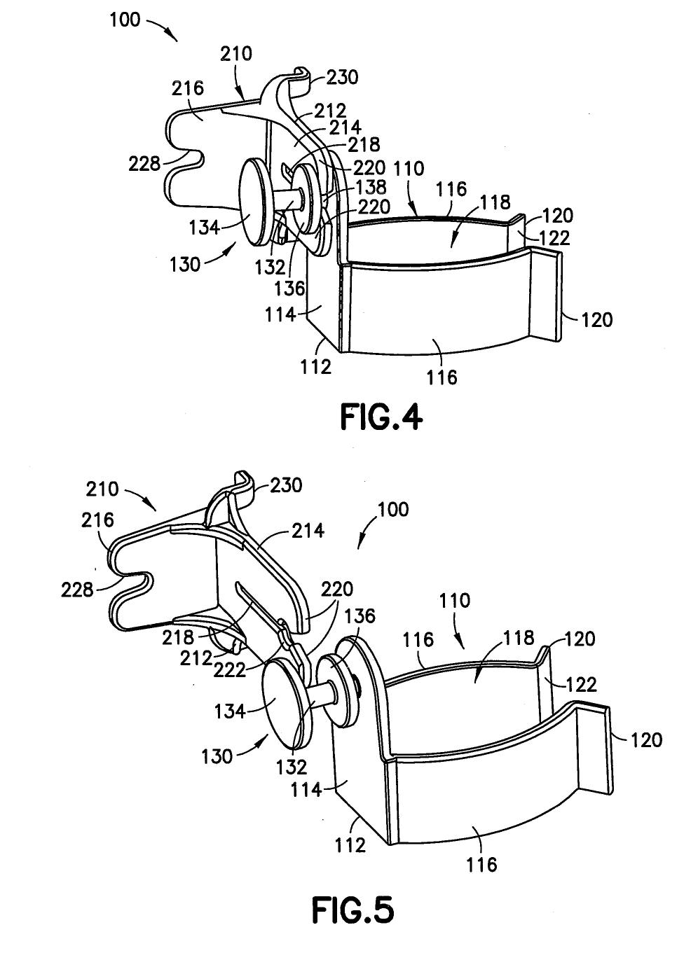

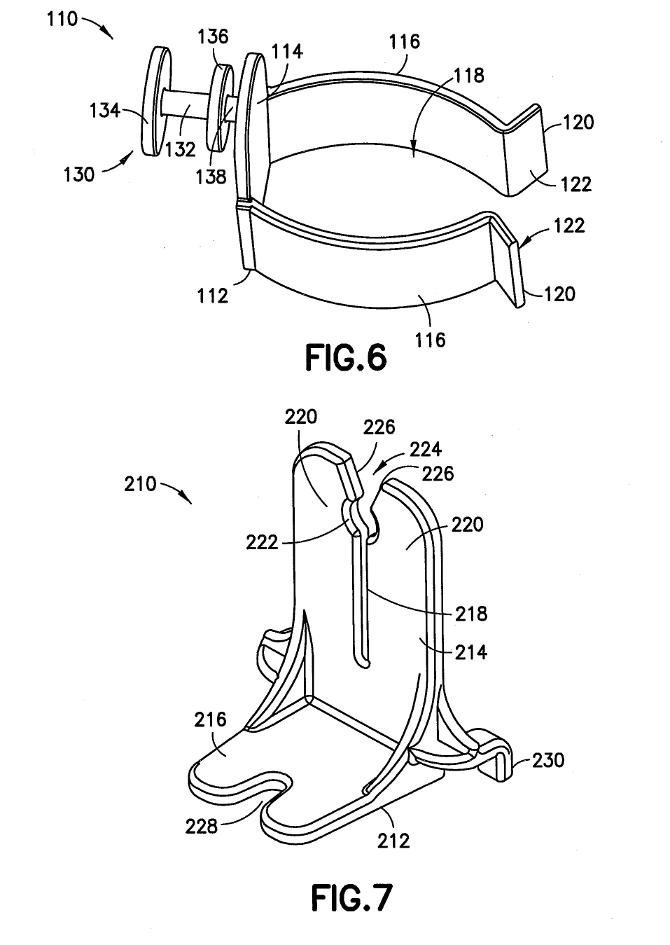

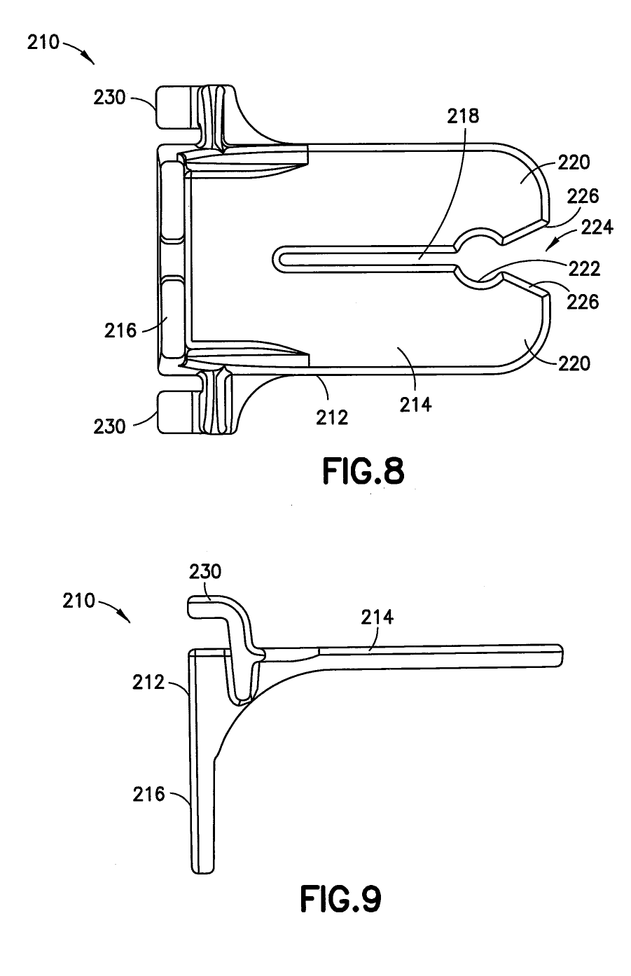

Referring further to FIGS. 4-10, an embodiment of a support assembly 100 for supporting the hand controller 40 in association with the fluid delivery system 1 and, in particular, in association with the fluid injector head 3 is shown. The support assembly 100 is comprised of a first component 110 and a second component 210. The first component 110 desirably comprises a unitary body 112 which is formed with a base portion or plate 114 and two (2) outward extending support arms 116 adapted to interface with the hand controller 40. The unitary body 112 may be made of a suitable grade of plastic for medical applications. In particular, the support arms 116 are generally arcuate-shaped to define a generally oval-shaped area or receiving space 118 therebetween for receiving and supporting the hand controller 40. The support arms 116 terminate in respective outward directed tip ends 120 so as to define outwardly tapered surfaces 122 so that the hand controller 40 may be easily placed between the support arms 116. The support arms 116 are spaced apart such that the opposing arms 60, 62 formed on the housing 42 of the hand controller 40 may rest upon the support arms 116 to support the hand controller 40. Thus, the support arms 116 provide supporting locations for the hand controller 40 when the hand controller 40 is present in the support assembly 100.

A rotational enabling assembly 130 extends proximally or rearward from the base portion 114 of the first component 110. The rotational enabling assembly 130 comprises a support post or shaft 132, a first or proximal button element 134, and a second or distal button element 136. A post portion 138 of the support post or shaft 132 between the distal button element 136 and the base portion or plate 114 provides an interface location or area for interfacing the first component 110 with the second component 210 as described herein. Further, the post portion 138 further permits the first component 110 to rotate relative to the second component 210, as described herein, such that the first component 110 can rotate in the support assembly 100 as the fluid injector head 3 rotates. The distal button element 136 limits axial movement of the support post or shaft 132. The foregoing rotational or swivel feature maintains the hand controller 40 upright in the support assembly 100 so that the hand controller 40 does not fall out of the support assembly 100 as the fluid injector head 3 is rotated into different rotational positions or orientations.

The second component 210 likewise desirably has a unitary body and is formed as an L-shaped body 212. The L-shaped body 212 may be made of a suitable grade of plastic for medical applications. The L-shaped body 212 comprises a first leg 214 and a second leg 216 defining approximately a 90° angle between one another. The first leg 214, which is the longer segment, defines a longitudinal slot 218 between two (2) arm portions 220. The longitudinal slot 218 is formed with a circular recess 222 that receives the post portion 138 of the support post 132, such that the post shaft 132 may rotate within the circular recess 222. The circular recess 222 is accessed via a V-shaped notch 224 defined by opposed tapered ends 226 of the arm portions 220. The tapered ends 226 restrain the support post 132 within the circular recess 222, with the support post 132 held in place in the circular recess 222 via a friction fit-snap fit connection. The post portion 138 is the portion of the support post 132 held in place within the circular recess 222, and the degree of friction fit-snap fit within the circular recess 222 determines how freely the first component 110 may rotate or swivel with respect to the second component 210 in the support assembly 100. Thus, when a hand controller 40 is supported in the first component 110, tightness or looseness of the friction fit-snap fit determines whether the first component 110 may automatically reposition itself under the force of gravity as the fluid injector head 3 is rotated to different angular positions, or whether an operator must manually reposition the first component 110 relative to the second component 210, which is typically fixed to the fluid injector head 3.

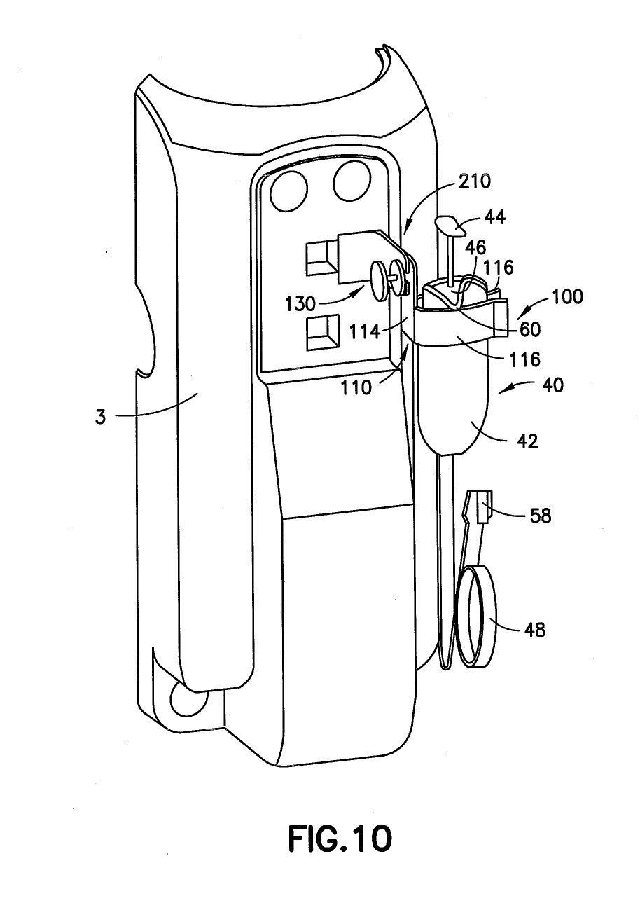

As noted, the second component 210 is adapted to be fixed to the fluid injector head 3. In particular, the second leg 216 of the second component 210 comprises a slot 228 adapted to accept a mechanical fastener and the like to secure the second component 210 to the fluid injector head 3 on a desired surface of the fluid injector head 3, such as the bottom surface or wall of the housing of the fluid injector head 3 as shown in FIG. 10. The second leg 216 may further comprise stabilizing legs 230 adapted to contact the fixing surface of the fluid injector head 3 to support the support assembly 100 on the fluid injector head 3.

As shown in FIG. 10, once the support assembly 100 is affixed to a suitable surface or face of the fluid injector head 3, the hand controller 40 may be placed between the support arms 116 of the first component 110. The support arms 116 support the opposing arms 60, 62 formed on the housing 42 of the hand controller 40 to support the hand controller 40 in association with the support assembly 100. Due to the rotational or swivel connection between the first component 110 and the second component 210, as the fluid injector head 3 is rotated to different angular orientations during use, the first component 110 may rotate relative to the second component 210 to maintain the hand controller 40 in an upright position and supported by the support assembly 100 during rotational movement of the fluid injector head 3. As noted in the foregoing, the hand controller 40 may utilize a sterile sheath 80, which is configured as a generally form-fitting envelope enclosing at least the housing 42 of the hand controller 40. The first component 110 is adapted to receive the hand controller 40 with or without a disposable sheath 80 and, thus, the support assembly 100 is multi-purpose assembly, which supports the hand controller 40 and, further, retains the sterile sheath 80 in place on the hand controller 40.

While several embodiments of a hand controller support assembly and fluid delivery system incorporating the same are shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A hand controller support assembly, comprising:
a first component comprising a base portion and two outward extending support arms to support the hand controller;
a second component adapted for fixation to a surface; and
wherein the first component is rotationally connected to the second component such that the first component may automatically reposition relative to the second component under the force of gravity to maintain the hand controller in an upright position and supported by the support assembly.

2. A hand controller support assembly as claimed in claim 1, wherein the second component comprises an L-shaped body comprising a first leg and a second leg defining approximately a 90° angle between one another.

3. A hand controller support assembly as claimed in claim 2, wherein the first leg is longer than the second leg.

4. A hand controller support assembly as claimed in claim 2, wherein the first leg defines a longitudinal slot between two arm portions, the longitudinal slot formed with a circular recess to receive a support post extending from the base portion of the first component to enable rotational movement of the first component relative to the second component.

5. A hand controller support assembly as claimed in claim 2, wherein the circular recess is accessed via a V-shaped notch defined by opposed tapered ends of the arm portions.

6. A hand controller support assembly as claimed in claim 1, wherein the second component comprises an L-shaped body comprising a first leg and a second leg, and wherein the second leg defines a slot for a fastener to attach the second component to the fixation surface.

7. A hand controller support assembly as claimed in claim 6, wherein the second leg further comprises stabilizing legs adapted to contact the fixation surface.

8. A hand controller support assembly as claimed in claim 1, further comprising a support post extending from the base portion of the first component.

9. A hand controller support assembly as claimed in claim 8, wherein the second component comprises an L-shaped body comprising a first leg and a second leg, and wherein the first leg defines a longitudinal slot between two arm portions, the longitudinal slot formed with a circular recess to receive the support post to enable rotational movement of the first component relative to the second component.

10. A hand controller support assembly as claimed in claim 9, wherein the support post comprises a distal button to limit axial movement of the support post in the circular recess.

11. A fluid delivery system, comprising:
a fluid injector head;
a hand controller electronically interfaced with the fluid injector head; and
a hand controller support assembly for supporting the hand controller, comprising:
a first component comprising a base portion and two outward extending support arms to support the hand controller;
a second component adapted for fixation to a surface; and
wherein the first component is rotationally connected to the second component such that the first component may automatically reposition relative to the second component under the force of gravity to maintain the hand controller in an upright position and supported by the support assembly.

12. A fluid delivery system as claimed in claim 11, wherein the second component comprises an L-shaped body comprising a first leg and a second leg defining approximately a 90° angle between one another.

13. A fluid delivery system as claimed in claim 12, wherein the first leg is longer than the second leg.

14. A fluid delivery system as claimed in claim 12, wherein the first leg defines a longitudinal slot between two arm portions, the longitudinal slot formed with a circular recess to receive a support post extending from the base portion of the first component to enable rotational movement of the first component relative to the second component.

15. A fluid delivery system as claimed in claim 12, wherein the circular recess is accessed via a V-shaped notch defined by opposed tapered ends of the arm portions.

16. A fluid delivery system as claimed in claim 11, wherein the second component comprises an L-shaped body comprising a first leg and a second leg, and wherein the second leg defines a slot for a fastener to attach the second component to the fixation surface.

17. A fluid delivery system as claimed in claim 16, wherein the second leg further comprise stabilizing legs adapted to contact the fixation surface.

18. A fluid delivery system as claimed in claim 11, further comprising a support post extending from the base portion of the first component.

19. A fluid delivery system as claimed in claim 18, wherein the second component comprises an L-shaped body comprising a first leg and a second leg, and wherein the first leg defines a longitudinal slot between two arm portions, the longitudinal slot formed with a circular recess to receive the support post to enable rotational movement of the first component relative to the second component.

20. A fluid delivery system as claimed in claim 19, wherein the support post comprises a button element to limit axial movement of the support post in the circular recess.
